(19) United States
(12) Patent Application Publication
(10) Pub. No.: US 2018/0325637 A1
(43) Pub. Date: Nov. 15, 2018

(54) ORAL CARE DEVICE FOR INTUBATED PATIENTS

(71) Applicant: Theodore D. Fox, Greeneville, TN (US)

(72) Inventor: Theodore D. Fox, Greeneville, TN (US)

(21) Appl. No.: 15/977,759

(22) Filed: May 11, 2018

Related U.S. Application Data
(60) Provisional application No. 62/504,804, filed on May 11, 2017.

(51) Int. Cl. A61C 17/06; A46B 9/02

(52) U.S. Cl. CPC A61C 17/043; A46B 9/025; A61M 16/04; A46B 2200/1066; A46B 9/028

(57) ABSTRACT

Example embodiments of the present general inventive concept provide an oral care device including a handle having a proximal end and a distal end, a suction connector provided at the proximal end of the handle and configured to be coupled to a suction apparatus, a plurality of bristles provided proximate the distal end of the handle and configured in a spiral pattern around a circumference of the distal end of the handle, and one or more suction ports provided proximate the distal end of the handle.

### CROSS-REFERENCE TO RELATED APPLICATIONS

This application claims the benefit of U.S. Provisional Patent Application Ser. No. 62/504,804, filed on May 11, 2017, which is incorporated herein in its entirety by reference.

### FIELD OF INVENTION

The present general inventive concept relates to an oral care device, and, more particularly, to an oral care device to increase efficiency and ease of use when providing oral care to a medical patient.

### BACKGROUND

In many medical care situations, a patient may be incapacitated or otherwise unable to provide his/her own oral care, leaving a nurse or other medical assistant responsible for providing such care. In some situations, such as when the patient has undergone intubation, access to the teeth of the patient for cleaning can be very difficult, because of the limited available space within the patient's mouth in which the assistant must maneuver a toothbrush or other cleaning device. Additionally, there have been recent recommendations that oral care should be performed every couple of hours or so on ventilator patients. Along with the other primary concerns for the patient, without proper oral care instances of pneumonia and other maladies may increase, causing increase costs for the hospital. Therefore, an oral care device that would be more easily maneuverable, and provide a more efficient method of cleaning, would be desirable.

### BRIEF SUMMARY

According to various example embodiments of the present general inventive concept, an oral care device is provided having spiral rows of bristles and at least one suction port to provide enhanced oral care for an incapacitated or reduced mobility patient.

Additional aspects and advantages of the present general inventive concept will be set forth in part in the description which follows, and, in part, will be obvious from the description, or may be learned by practice of the present general inventive concept.

The foregoing and/or other aspects and advantages of the present general inventive concept may be achieved by an oral care device including a handle having a proximal end and a distal end, a suction connector provided at the proximal end of the handle and configured to be coupled to a suction apparatus, a plurality of bristles provided proximate the distal end of the handle and configured in a spiral pattern around a circumference of the distal end of the handle, and one or more suction ports provided proximate the distal end of the handle.

The handle may be configured as a hollow tube between the suction connector and the one or more suction ports.

At least one of the one or more suction ports may be provided on the distal end of the handle in linear alignment with the handle.

At least one of the one or more suction ports may be provided at a side of the handle between adjacent rows of the bristles.

The rows of the bristles may be spaced apart such that the at least one of the one or more suction ports is smaller than a space between the rows.

A plurality of suction ports may be respectively provided between adjacent rows of the bristles.

The plurality of suction ports may be provided at at least two different circumferential locations of the distal end of the handle.

The bristles may extend in a substantially perpendicular direction from the handle.

At least a portion of the bristles may extend at an acute angle relative to the handle.

At least a first quantity of the bristles may extend at an acute angle relative to the handle in a direction of the proximal end, and at least a second quantity of the bristles may extend at an acute angle relative to the handle in a direction of the distal end.

Other features and aspects may be apparent from the following detailed description, the drawings, and the claims.

### BRIEF DESCRIPTION OF THE FIGURES

The following example embodiments are representative of example techniques and structures designed to carry out the objects of the present general inventive concept, but the present general inventive concept is not limited to these example embodiments. In the accompanying drawings and illustrations, the sizes and relative sizes, shapes, and qualities of lines, entities, and regions may be exaggerated for clarity. A wide variety of additional embodiments will be more readily understood and appreciated through the following detailed description of the example embodiments, with reference to the accompanying drawings in which:

FIGS. 1A-1B illustrate items typically found in a conventional oral care kit for a medical patient;

FIGS. 2A-2D illustrate oral care devices according to examples of the present general inventive concept; and

FIGS. 3A-3C and 4A-4B illustrate various features of example embodiments of the present general inventive concept.

### DETAILED DESCRIPTION

Reference will now be made to the example embodiments of the present general inventive concept, examples of which are illustrated in the accompanying drawings and illustrations. The example embodiments are described herein in order to explain the present general inventive concept by referring to the figures.

The following detailed description is provided to assist the reader in gaining a comprehensive understanding of the structures and fabrication techniques described herein. Accordingly, various changes, modification, and equivalents of the structures and fabrication techniques described herein will be suggested to those of ordinary skill in the art. The progression of fabrication operations described are merely examples, however, and the sequence type of operations is not limited to that set forth herein and may be changed as is known in the art, with the exception of operations necessarily occurring in a certain order. Also, description of well-known functions and constructions may be simplified and/or omitted for increased clarity and conciseness.

Note that spatially relative terms, such as “up,” “down,” “right,” “left,” “beneath,” “below,” “lower,” “above,” “upper” and the like, may be used herein for ease of description to describe one element or feature's relationship to another element(s) or feature(s) as illustrated in the figures. Spatially relative terms are intended to encompass different orientations of the device in use or operation in addition to the orientation depicted in the figures. For example, if the device in the figures is turned over or rotated, elements described as “below” or “beneath” other elements or features would then be oriented “above” the other elements or features. Thus, the exemplary term “below” can encompass both an orientation of above and below. The device may be otherwise oriented (rotated 90 degrees or at other orientations) and the spatially relative descriptors used herein interpreted accordingly.

According to various examples of the present general inventive concept, an oral care device is configured with spiral rows of bristles and at least one suction port to provide enhanced oral care for an incapacitated or otherwise reduced mobility patient. For example, an oral care device may be provided that includes a handle having a proximal end and a distal end, a suction connector provided at the proximal end of the handle and configured to be coupled to a suction apparatus, a plurality of bristles provided proximate the distal end of the handle and configured in a spiral pattern around a circumference of the distal end of the handle, and one or more suction ports provided proximate the distal end of the handle.

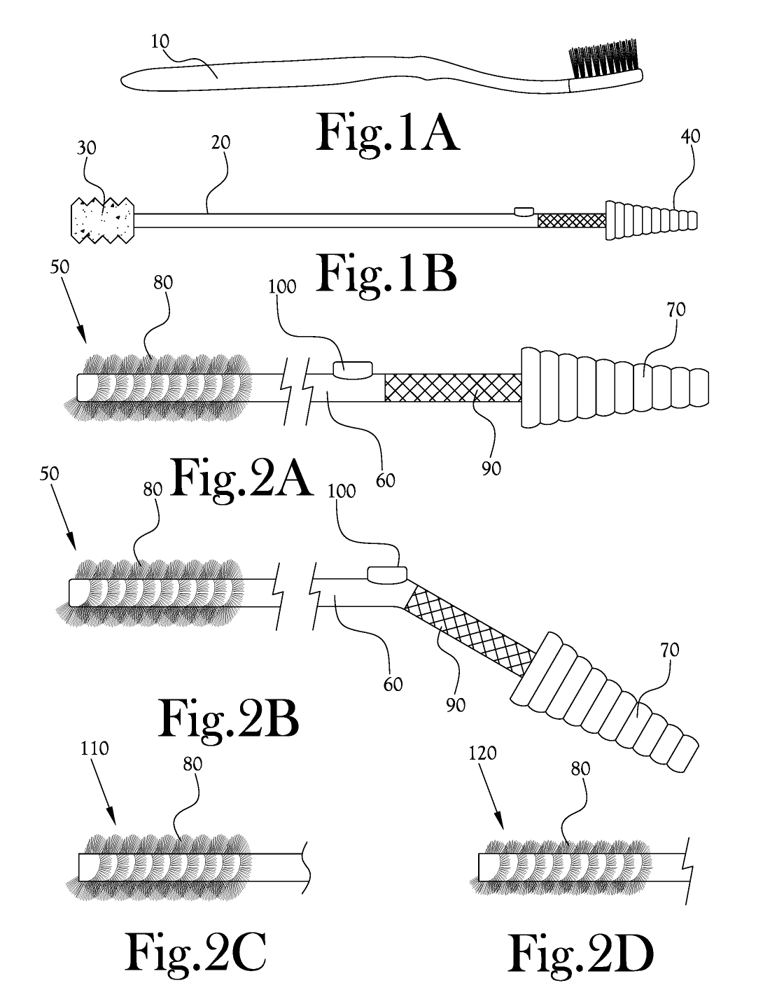

FIGS. 1A-1B illustrate items typically found in a conventional oral care kit for a medical patient. For patients that are incapacitated or medically inconvenienced enough to require assistance in oral care, such as the brushing of the patient's teeth, a conventional oral kit may be provided which has a standard toothbrush 10 as illustrated in FIG. 1A, and a standard sponge-suction swab 20 as illustrated in FIG. 1B. While the standard toothbrush 10 of FIG. 1A may be more capable of providing a thorough cleaning of the patient's teeth and gums, many healthcare workers typically opt instead for the sponge-suction swab 20 due to the relatively easier manipulation and cleaning, as well as the shorter time required for cleaning. The ease of using the swab 20 is due to factors such as a sponge 30 being provided around 360 degrees of the distal end of the sponge-suction swab 20, and the suction provided through sponge 30. As illustrated in FIG. 1B, the sponge 30 extends around the circumference of the handle, as well as a distance around the distal end, and is therefore much easier to manipulate in the mouth of a patient, especially a patient that is intubated or has other such medical equipment that cannot be easily removed from the patient's mouth. Also, as illustrated in FIG. 1B, a suction connector 40 may be provided to the sponge-suction swab 20 such that suction can be actuated and draw excess liquids from the mouth of the patient through the swab head. However, the tradeoff for such convenience for the healthcare assistant is that the cleaning leaves much to be desired when using only the sponge-suction swab 20, especially in comparison to the standard toothbrush 10, which may often be simply discarded.

Therefore, a goal and feature of the present general inventive concept is to provide an oral care device that improves a thoroughness and efficiency of cleaning, and also increases the convenience of use for the healthcare assistant using the device. FIGS. 2A-2D illustrate oral care devices according to examples of the present general inventive concept. The oral care device 50 in the example embodiment illustrated in FIG. 2A has a main body portion which may be referred to as the handle 60, which is generally much of the portion of the device 50 between the proximal and distal ends. At the proximal end of the device 50 is a suction connector 70 that may be connected to a suction hose or other suction device to provide suction action to the oral care device 50. In various example embodiments, and as illustrated in FIGS. 2A-2B, the suction connector 70 may be a male adapter end configured to be inserted into a female end of standard medical suction tubing. At the distal end of the device are rows of bristles 80 that are arranged in a substantially spiral shape repeating around the circumference of the distal end of the device 50. In various example embodiments the spiral rows of bristles 80 may be provided with a substantially constant sized gap between the rows, similar to a rifling pattern. In other example embodiments the gaps between the spiral rows of bristles may increase, decrease, or otherwise vary as the distance from the distal end of the device 50 increases. In some example embodiments of the present general inventive concept, the bristles 80 may extend from the circumference of the device 50 in an arrangement substantially perpendicular to the surface of the device. In other example embodiments, the bristles may vary in the angles relative to the surface of the device 50. For example, some or all of the bristles 80 may be angled toward the proximal or distal end of the device, the bristles 80 may be arranged such that different areas of bristles 80 extend in different directions, and so on. In various example embodiments, the bristles 80 may be arranged in tufts. According to various example embodiments of the present general inventive concept, the bristles 80 may be standard monofilament bristles. As discussed later in these descriptions, in various example embodiments suction ports may be provided between rows of bristles 80 and/or on the distal end of the device 50.

In various example embodiments of the present general inventive concept, a textured handle portion 90 may be provided proximate the proximal end of the device, as illustrated in FIGS. 2A-2B. The textured handle portion 90 provides a more secure grip to the healthcare assistant when the device 50 becomes wet during oral care of the patient. In various example embodiments, as illustrated in FIG. 2A, a portion of the proximal end including the textured handle portion 90 and suction connector 70 extends in the same line formed by the handle 60 of the device 50. In other various example embodiments, a portion of the proximal end including the textured handle portion 90 and suction connector 70 may be angled away from the rest of the handle 60 of the device 50 to aid in the convenience of the healthcare assistant providing the oral care. In other words, the angled portion may be provided such that the arm and/or wrist of the healthcare assistant will not have to be held so high while providing the oral care. Also, as illustrated in FIGS. 2A-2B, the device may be provided with a suction actuator 100 or initiator to cause the suction provided at the suction connector 70 to be provided at the suction port(s) at the distal to remove excess fluids from the mouth of the patient. For example, the suction actuator 100 may be a button that is easily accessed by the thumb of the healthcare assistant, and the pressing of the button 100 may open the hollow portion of the handle 60 provided between the suction connector 70 and the suction port(s). In other example embodiments, the suction actuator 100 may simply be an aperture which negates suction at the distal end of the device 50 unless the healthcare assistant covers the aperture with a thumb, thereby allowing the suction at the suction connector 70 to cause suction at the suction port(s).

According to various example embodiments of the present general inventive concept, the length of the spirally configured bristles 80 may be configured according to the needs of the patient. FIG. 2C illustrates a spiral brush head 110 configured for normal use with adults, while FIG. 2C illustrates a spiral brush head 120 configured for pediatric patients, wherein the bristles are considerably shorter for easier manipulation in the smaller mouths of children. In various example embodiments of the present general inventive concept, the bristles on the adult spiral brush head 110 may extend approximately 0.5 inches from the handle 60, and the bristles on the pediatric spiral brush head 120 may extend approximately 0.3 inches from the handle 60.

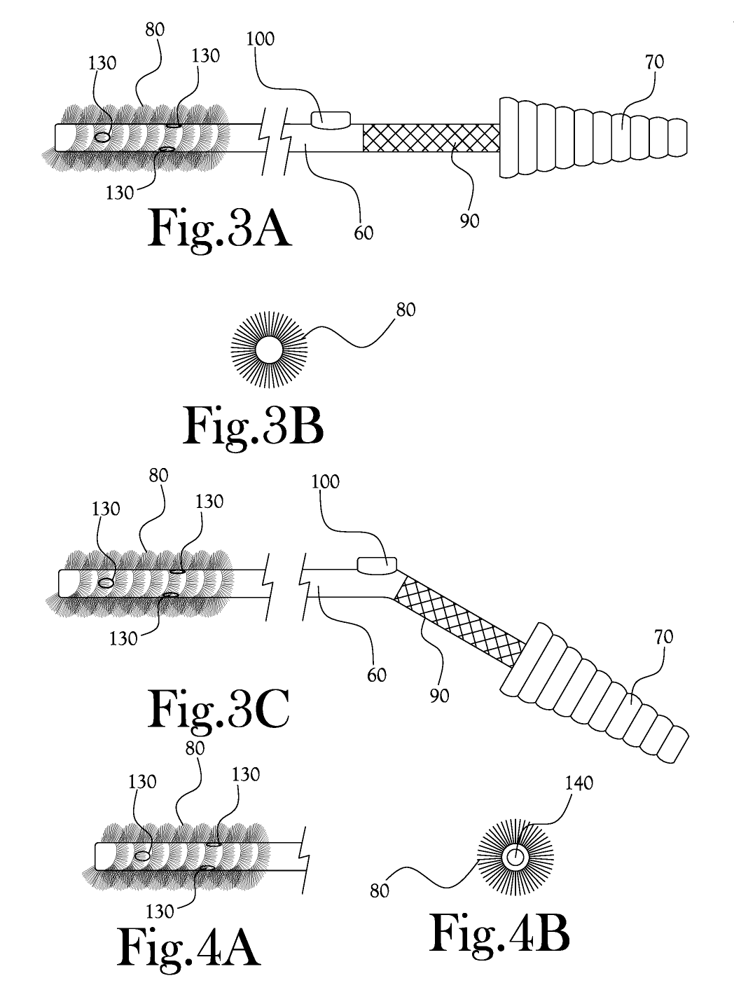

FIGS. 3A-3C and 4A-4B illustrate various features of example embodiments of the present general inventive concept. The configurations and sizes of the features illustrated in FIGS. 3A-3C and 4A-4B are merely examples of features which may be provided to the device of the present general inventive concept, and it is understood that the device is not limited to any combination or arrangement of features illustrated therein. As illustrated in FIG. 3A, the rifled or spiral bristles 80 are configured to wrap around 360 degrees of the device several times. In various example embodiments, such as the example of a pediatric brush illustrated in FIGS. 3A-3B, the bristles 80 may have a length of 0.3 to 0.5 inches, and may be provided along a length of 1.0 to 1.25 inches of the distal end of the device 50. In various examples of the adult device according to the present general inventive concept, such as the embodiment illustrated in FIG. 3C, the bristles 80 may have a length of approximately 0.5 inches. In various example embodiments, a suction port 140 may be provided at the distal end of the device 50, configured as an opening on the face of the distal end, facing the axial direction of the handle 60. Such a feature is illustrated in FIGS. 3A and 4A, and in more detail in FIGS. 3B and 4B.

In various example embodiments of the present general inventive concept, one or more suction ports 130 may be provided between the spiral rows of bristles 80. The spiral configuration of the bristles 80 may be configured such that the distance between adjacent rows of bristles 80 is larger than the diameter of the suction ports 130 provided therebetween. As illustrated in FIG. 3A, a plurality of suction ports 130 may be provided and configured such that at least some of the suction ports 130 do not face the same direction. In other words, the suction ports 130 may be located at different circumferential positions about the distal end of the oral care device. However, the more suction ports 130 that are provided, the more the overall suction power of the device may be reduced. Therefore, in various example embodiments the circumferential suction ports may only include two such ports configured 180 degrees from one another, or only one port between the rows of bristles 80. Various example embodiments of the present general inventive concept may only have one or more of the laterally facing suction ports 130, while other various example embodiments may only have the axially facing suction port 140. Various example embodiments may have both the axially facing suction port 140 and the one or more laterally facing suction ports 130.

According to various example embodiments of the present general inventive concept, an oral care device is provided including a handle having a proximal end and a distal end, a suction connector provided at the proximal end of the handle and configured to be coupled to a suction apparatus, a plurality of bristles provided proximate the distal end of the handle and configured in a spiral pattern around a circumference of the distal end of the handle, and one or more suction ports provided proximate the distal end of the handle. The handle may be configured as a hollow tube between the suction connector and the one or more suction ports. At least one of the one or more suction ports may be provided on the distal end of the handle in linear alignment with the handle. At least one of the one or more suction ports may be provided at a side of the handle between adjacent rows of the bristles. The rows of the bristles may be spaced apart such that the at least one of the one or more suction ports is smaller than a space between the rows. A plurality of suction ports may be respectively provided between adjacent rows of the bristles. The plurality of suction ports may be provided at at least two different circumferential locations of the distal end of the handle. The bristles may extend in a substantially perpendicular direction from the handle. At least a portion of the bristles may extend at an acute angle relative to the handle. At least a first quantity of the bristles may extend at an acute angle relative to the handle in a direction of the proximal end, and at least a second quantity of the bristles may extend at an acute angle relative to the handle in a direction of the distal end. Various other example embodiments may provide bristles that extend in one or more other directions, such as an acute angle relative to the handle that extends toward neither the proximal nor distal ends, or a combination of several different angles and/or directions. With such a device, a healthcare assistant may provide better and more thorough oral care with the same or similar time and effort used conventionally with the device illustrated in FIG. 1B.

Numerous variations, modifications, and additional embodiments are possible, and accordingly, all such variations, modifications, and embodiments are to be regarded as being within the spirit and scope of the present general inventive concept. For example, regardless of the content of any portion of this application, unless clearly specified to the contrary, there is no requirement for the inclusion in any claim herein or of any application claiming priority hereto of any particular described or illustrated activity or element, any particular sequence of such activities, or any particular interrelationship of such elements. Moreover, any activity can be repeated, any activity can be performed by multiple entities, and/or any element can be duplicated.

It is noted that the simplified diagrams and drawings included in the present application do not illustrate all the various connections and assemblies of the various components, however, those skilled in the art will understand how to implement such connections and assemblies, based on the illustrated components, figures, and descriptions provided herein, using sound engineering and medical judgment. Numerous variations, modification, and additional embodiments are possible, and, accordingly, all such variations, modifications, and embodiments are to be regarded as being within the spirit and scope of the present general inventive concept.

While the present general inventive concept has been illustrated by description of several example embodiments, and while the illustrative embodiments have been described in detail, it is not the intention of the applicant to restrict or in any way limit the scope of the general inventive concept to such descriptions and illustrations. Instead, the descriptions, drawings, and claims herein are to be regarded as illustrative in nature, and not as restrictive, and additional embodiments will readily appear to those skilled in the art upon reading the above description and drawings. Additional modifications will readily appear to those skilled in the art. Accordingly, departures may be made from such details without departing from the spirit or scope of applicant's general inventive concept.

1. An oral care device, comprising:
a handle having a proximal end and a distal end;
a suction connector provided at the proximal end of the handle and configured to be coupled to a suction apparatus;
a plurality of bristles provided proximate the distal end of the handle and configured in a spiral pattern around a circumference of the distal end of the handle; and
one or more suction ports provided proximate the distal end of the handle.

2. The oral care device of claim 1, wherein the handle is configured as a hollow tube between the suction connector and the one or more suction ports.

3. The oral care device of claim 1, wherein at least one of the one or more suction ports is provided on the distal end of the handle in linear alignment with the handle.

4. The oral care device of claim 1, wherein at least one of the one or more suction ports is provided at a side of the handle between adjacent rows of the bristles.

5. The oral care device of claim 4, wherein the rows of the bristles are spaced apart such that the at least one of the one or more suction ports is smaller than a space between the rows.

6. The oral care device of claim 4, wherein a plurality of suction ports are respectively provided between adjacent rows of the bristles.

7. The oral care device of claim 6, wherein the plurality of suction ports are provided at at least two different circumferential locations of the distal end of the handle.

8. The oral care device of claim 1, wherein the bristles extend in a substantially perpendicular direction from the handle.

9. The oral care device of claim 1, wherein at least a portion of the bristles extend at an acute angle relative to the handle.

10. The oral care device of claim 1, wherein at least a first quantity of the bristles extend at an acute angle relative to the handle in a direction of the proximal end, and wherein at least a second quantity of the bristles extend at an acute angle relative to the handle in a direction of the distal end.
